(19) United States
(12) Patent Application Publication
(10) Pub. No.: US 2012/0040908 A1
(43) Pub. Date: Feb. 16, 2012

(54) FRACTURE REPAIR PROMOTER

(75) Inventors: Mizuho Kido, Fukuoka (JP); Toshiyuki Watanabe, Fukuoka (JP); Atsushi Danjo, Fukuoka (JP); Teruo Tanaka, Fukuoka (JP); Daisuke Uetsuji, Hokkaido (JP); Aiko Ono, Hokkaido (JP); Atsushi Serizawa, Hokkaido (JP)

(73) Assignees: MEGMILK SNOW BRAND CO., LTD., Hokkaido (JP); KYUSHU UNIVERSITY, NATIONAL UNIVERSITY CORP., Fukuoka (JP)

(21) Appl. No.: 13/129,613

(22) PCT Filed: Oct. 19, 2009
(86) PCT No.: PCT/JP2009/068295

§ 371 Date: Sep. 8, 2011

(30) Foreign Application Priority Data
Nov. 18, 2008 (JP) 2008-294445

(51) Int. Cl. A61K 38/17; A61P 19/00; A61P 19/08

(52) U.S. Cl. 514/16.7

(57) ABSTRACT

Disclosed is a substance capable of promoting the repair of a fractured part in a fracture caused by an external force, fatigue or a disease. Also disclosed is a method for producing the substance. Further disclosed is a product capable of promoting the repair of a fracture, such as a food, a beverage and a feed, which comprises the fracture repair promoter. Specifically disclosed is a fracture repair promoter comprising, as an active ingredient, a fraction containing a milk-derived basic protein. In the promoter, the fraction containing the milk-derived basic protein is produced by contacting milk or a milk-derived raw material with a cation exchange resin to cause the adsorption of the basic protein on the cation exchange resin and eluting a fraction adsorbed on the resin by means of an eluent having a salt concentration of 0.1 to 1.0 M.

### TECHNICAL FIELD

The present invention relates to a fracture repair promoter, a method of producing the same, and a food, drink, or feed comprising the fracture repair promoter. The fracture repair promoter has an effect to promote multistage reactions such as inflammation, chondrogenesis or subperiosteal bone formation, vascularization, and bone remodeling which are fracture repair reactions. Therefore, the fracture repair promoter is useful for treating a fracture.

### BACKGROUND ART

In recent years, the risk associated with bone diseases (e.g., osteoporosis and fracture) tends to increase along with aging. Bone formation by osteoblasts and bone resorption by osteoclasts are constantly well-balanced in bone tissues. Osteoporosis however occurs when the balance between bone formation and bone resorption has not been kept, and bone resorption has become predominant. In particular, the function of osteoclasts that causes bone resorption becomes predominant in elderly women due to insufficient estrogen secretion after the menopause. It is necessary to take measures that maintain the bone mass in order to prevent osteoporosis. A vitamin D preparation and the like have been disclosed as a medical drug that alleviates a loss of bone mass and the fracture incidence due to osteoporosis.

However, some tests conducted on elderly persons suggest that it is not clear whether or not the vitamin D preparation promotes repair of a fracture in a person with a sufficient quantity of vitamin D (see Non-patent Documents 1 and 2, for example). A fracture is repaired through process steps of inflammation, callus formation, collagen production by chondrocytes in callus, vascularization, and bone remodeling. On the other hand, bone formation is performed using the function of osteoblasts, and plays only part of the fracture repair process. Examples of a factor that affects differentiation and growth of osteoblasts include cbfa-1, FGF-1, FGF-2, a milk-derived basic protein fraction, and the like (see Patent Document 1 and Non-patent Document 3, for example). However, the fracture repair process involves complex reactions in bone tissues including blood vessels and nerves. Therefore, it is unclear whether or not the fracture repair process that involves complex reactions can be promoted by merely promoting bone formation by osteoblasts. For example, FGF-2 promotes the growth of osteoblasts, but adversely affects differentiation of osteoblasts and collagen production in chondrocytes (see Non-patent Document 4, for example).
[Patent Document 1] JP-A-H08-151331
[Non-patent Document 1] Paul Lips et al., Vitamin D supplementation and fracture incidence in elderly persons a randomized, placebo-controlled clinical trial, Annals of Internal Medicine, 15 Feb., 1996, 124(4), p. 400-406
[Non-patent Document 2] M. Law et al., Vitamin D supplementation and the prevention of fractures and falls: results of a randomized trial in elderly people in residential accommodation. Age Ageing, 1 Sep., 2006, 35(5), p. 482-486
[Non-patent Document 3] Frederic Shapiro, Bone development and its relation to fracture repair. The role of mesenchymal osteoblasts and surface osteoblasts. European Cells and Materials, Vol. 15, 2008, p. 53-76
[Non-patent Document 4] Takashi Shimoaka et al., Regulation of Osteoblast, Chondrocyte, and Osteoclast Functions by Fibroblast Growth Factor (FGF)-18 in Comparison with FGF-2 and FGF-10. The Journal of Biological chemistry, Vol. 277, No. 9, Mar. 1, 2002, p. 7493-7500

### DISCLOSURE OF THE INVENTION

### Problems to be Solved by the Invention

A bone fracture occurs when the bone cannot withstand the outside force, and the fracture site is then subsequently repaired through process steps of inflammation, callus formation, vascularization, and bone remodeling. Specifically, the fracture repair process involves complex reactions in bone tissues including blood vessels and nerves. Therefore, it is unclear whether or not the fracture repair process that involves complex reactions can be promoted by merely promoting bone formation due to osteoblasts. Repair of a fracture may not be promoted even if the above drug or the like is administered. Specifically, the above substance merely has a pharmacological effect on bone formation, and it is unclear whether or not the above substance promotes a series of fracture repair reactions. Therefore, fracture repair is not promoted even though a substance administered has a bone formation effect, but insofar as the substance does not have fracture repair effects relating to chondrocyte collagen production effect etc. together with the bone formation effect.

A bone fracture treatment is generally carried out as follows. When a simple fracture has occurred, the fracture site is fixed using an instrument. When a bone dislocation has occurred, the bone is returned to the normal position by taxis such as traction or operation. The bone is then maintained under a fixation or rest period of at least 3 weeks or 1 month. Since fracture repair thus takes time, a better fracture repair promoter may promote the fracture repair and shorten the period to be maintained the fixation and kept at rest, and consequently the burden imposed on the patient and the medical cost can be reduced. A fracture repair promoter that can be conveniently taken orally has not been proposed until now, and has been strongly desired for fracture patients and medical treatment.

In view of the above situation on promoting fracture repair, the inventors of the invention have extensively searched for a substance that is contained in a food material and exhibits a fracture repair-promoting effect. As a result, the inventors found that a milk-derived basic protein fraction or a basic peptide fraction obtained by hydrolyzing the milk-derived basic protein fraction using a protease, such as pepsin, pancreatin or the like can promote fracture repair through oral intake. The inventors also found that the basic protein fraction or the peptide fraction can be used as an active ingredient for a fracture repair promoter or a fracture repair-promoting food, drink, or feed. These findings have led to completion of the invention.

Accordingly, an object of the invention is to provide a fracture repair promoter that promotes repair of a fracture site through oral intake, a method of producing the same, and a food, drink, or feed that comprises the fracture repair promoter.

### Means for Solving the Problems

Specifically, the invention is as follows:

(1) A fracture repair promoter including a milk-derived basic protein fraction as an active ingredient.

(2) The fracture repair promoter according to (1), wherein the milk-derived basic protein fraction includes basic amino acids in an amount of 15 wt % or more to the total amino acids.

(3) A fracture repair promoter including a basic peptide fraction obtained by hydrolyzing the milk-derived basic protein fraction according to (1) or (2) using a protease as an active ingredient.

(4) The fracture repair promoter according to (3), wherein the protease is at least one protease selected from the group consisting of pepsin, trypsin and chymotrypsin.

(5) The fracture repair promoter according to (3), wherein the protease is at least one protease selected from the group consisting of pepsin, trypsin and chymotrypsin, and pancreatin.

(6) A food or drink including the milk-derived basic protein fraction or the basic peptide fraction according to any one of (1) to (5).

(7) A feed including the milk-derived basic protein fraction or the basic peptide fraction according to any one of (1) to (5).

(8) A method of producing a fracture repair promoter comprising bringing milk or a milk-derived raw material into contact with a cation-exchange resin to adsorb basic proteins on the cation-exchange resin, eluting a fraction adsorbed on the cation-exchange resin using an eluant having a salt concentration of 0.1M to 1.0M, and using the eluted fraction as an active ingredient.

(9) A method of producing a fracture repair promoter comprising bringing milk or a milk-derived raw material into contact with a cation-exchange resin to adsorb basic proteins on the cation-exchange resin, eluting a fraction adsorbed on the cation-exchange resin using an eluant having a salt concentration of 0.1M to 1.0M, hydrolyzing the eluted fraction using a protease, and using the fraction obtained through hydrolyzing step as an active ingredient.

(10) The method according to (9), wherein the protease is at least one protease selected from the group consisting of pepsin, trypsin and chymotrypsin.

(11) The method according to (9), wherein the protease is at least one protease selected from the group consisting of pepsin, trypsin and chymotrypsin, and pancreatin.

### Effects of the Invention

The fracture repair promoter according to the invention remarkably promotes repair of a fracture site, and is useful for treating a fracture caused by external force, a disease, or fatigue. The fracture repair promoter according to the invention can be conveniently taken orally. Since the fracture repair promoter according to the invention is derived from milk, the fracture repair promoter can be safely taken.

### BRIEF DESCRIPTION OF THE DRAWINGS

FIG. 1 shows photographs of mouse fracture models in a basic protein fraction non-administration group (CTRL) and a 1% basic protein fraction administration group (after 4 weeks from the operation) (Test Example 5).

FIG. 2 shows μCT images of mouse fracture models (Test Example 5).

FIG. 3 shows the total energy that indicates the toughness of a bone (Test Example 5).

### BEST MODE FOR CARRYING OUT THE INVENTION

A fracture repair promoter according to the invention is characterized in that a milk-derived basic protein fraction, or a basic peptide fraction obtained by hydrolyzing the basic protein fraction using a protease, is contained as an active ingredient. The milk-derived basic protein fraction may be obtained from mammalian milk such as cow's milk, human milk, goat's milk, or ewe's milk. The basic peptide fraction is obtained by acting with a protease on the milk-derived basic protein fraction. The milk-derived basic protein fraction and the basic peptide fraction have a function to promote repair of a fracture site. A fracture treatment can be fastened due to the above effect.

The milk-derived basic protein fraction used as the active ingredient of the fracture repair promoter has the following properties.

1) The milk-derived basic protein fraction includes several types of proteins having a molecular weight determined by sodium dodecyl sulfate polyacrylamide gel electrophoresis (SDS-PAGE) of 3000 to 80,000.

2) The milk-derived basic protein fraction includes proteins in an amount of 95 wt % or more, and includes a small amount of fats and ashes.

3) The proteins are mainly lactoferrin and lactoperoxidase.

4) The milk-derived basic protein fraction includes basic amino acids such as lysine, histidine, arginine and the like in an amount of 15 wt % or more to the total amino acids.

These basic protein fraction may be obtained by, for example, bringing a milk-derived raw material, such as skimmed milk, milk serum or the like, into contact with a cation-exchange resin so that basic proteins are adsorbed on the cation-exchange resin, eluting the basic protein fraction adsorbed on the cation-exchange resin using an eluant having a salt concentration of 0.1M to 1.0M, collecting the eluted fraction, desalting and concentrating the collected fraction using a reverse osmosis (RO) membrane, electrodialysis (ED) or the like, and optionally drying the resulting product.

As methods of obtaining the milk-derived basic protein fraction, the method of obtaining by bringing milk or a milk-derived raw material into contact with a cation exchanger to adsorb the basic proteins, and then eluting the basic protein fraction adsorbed on the cation exchanger using an eluant having a pH of more than 5 and an ionic strength of more than 0.5 (JP-A-H05-202098), or the method of obtaining by utilizing an alginic acid gel (JP-A-61-246198), the method of obtaining from a milk serum using porous inorganic particles (JP-A-1-86839), the method of obtaining from milk using a sulfate compound (JP-A-63-255300), and the like have been known. A basic protein fraction obtained by such methods may be used in the invention.

The milk-derived basic peptide fraction has the same amino acid composition as that of the basic protein fraction. For example, a peptide composition having an average molecular weight of 4000 or less may be obtained by treating a milk-derived basic protein fraction obtained by the above methods using a protease such as pepsin, trypsin, chymotrypsin or the like, and further optionally treating the resulting product using a protease such as pancreatin or the like.

The milk-derived basic protein fraction or the basic peptide fraction of an active ingredient may be administered as it is when administering the fracture repair promoter of the invention. Note that the milk-derived basic protein fraction or the basic peptide fraction may be used after preparing a drug product such as a powdered drug, granules, a tablet, a capsule, a thinkable preparation, or the like by a normal method. Since the milk-derived basic protein fraction or the basic peptide fraction is relatively stable against heat, a raw material including the milk-derived basic protein fraction or the basic peptide fraction can be heat-sterilized under conditions usually performed.

The dosage of the fracture repair promoter of the invention is determined taking account of the age, therapeutic effect, pathological condition, and the like, but may be normally about 10 to 500 mg/day. The fracture repair promoter of the invention may be formulated in food, drink, or feed so that the above dosage is ensured. The milk-derived basic protein fraction or the basic peptide fraction of the invention is not observed acute toxicity in rats. It is desirable that the milk-derived basic protein fraction or the basic peptide fraction of the invention be orally administered together with a calcium salt that exhibits excellent absorption. Examples of such a calcium salt may include calcium chloride, calcium carbonate, calcium lactate, an eggshell, a milk-derived calcium-containing substance, and the like.

### EXAMPLE

The invention is further described below in detail by way of examples and test examples. Note that these are merely exemplified, but should not be construed as limiting the invention.

### Example 1

A column (diameter: 5 cm, height: 30 cm) filled with 400 g of sulfonated Chitopearl (cation-exchange resin; manufactured by Fuji Spinning Co., Ltd.) was sufficiently washed with deionized water. 40 liters of unsterilized skimmed milk (pH: 6.7) was passed through the column at a flow rate of 25 ml/min. The column was then sufficiently washed with deionized water, and the basic protein fraction adsorbed on the resin was eluted with a 0.02M carbonate buffer solution (pH: 7.0) containing 0.98M sodium chloride. The eluate was desalted and concentrated using a reverse osmosis (RO) membrane, and freeze-dried to obtain 21 g of powdery basic protein fraction. The basic protein fraction may be directly used as the fracture repair promoter of the invention.

### Test Example 1

The molecular weight of the basic protein fraction obtained in Example 1 was determined by sodium dodecyl sulfate polyacrylamide gel electrophoresis (SDS-PAGE). The molecular weight was distributed in the range of 3000 to 80,000.

### Test Example 2

The composition of the basic protein fraction obtained in Example 1 was analyzed. The results are shown in Table 1. As shown in Table 1, the basic protein fraction mainly contained proteins.

TABLE 1

| Water | 1.06 (wt %) |
|---|---|
| Protein | 96.50 |
| Fat | 0.56 |
| Ash | 0.27 |
| Others | 1.61 |

### Test Example 3

The protein composition of the basic protein fraction obtained in Example 1 was analyzed. The results are shown in Table 2. The basic protein fraction contained lactoferrin and lactoperoxidase in amounts of 40 wt % or more, respectively.

TABLE 2

| Lactoferrin | 42.5 (wt %) |
|---|---|
| Lactoperoxidase | 45.6 |
| Insulin-like growth factor-1 | 0.005 |
| Others | 11.895 |

### Test Example 4

The basic protein fraction obtained in Example 1 was hydrolyzed at 110° C. for 24 hours using 6N hydrochloric acid, and the amino acid composition thereof was analyzed using an amino acid analyzer (“L-8500” manufactured by Hitachi Ltd.). The results are shown in Table 3. The basic protein fraction contained basic amino acids in an amount of 15 wt % or more to the total amino acids.

TABLE 3

| Aspartic acid | 10.1 (wt %) |
|---|---|
| Serine | 5.3 |
| Glutamic acid | 12.3 |
| Proline | 4.7 |
| Alanine | 5.7 |
| Leucine | 10.2 |
| Lysine | 8.4 |
| Histidine | 2.5 |
| Arginine | 7.2 |
| Others | 33.6 |

### Example 2

A column (diameter: 100 cm, height: 10 cm) filled with 30 kg of SP Toyopearl (cation-exchange resin, manufactured by Tosoh Corp.) was sufficiently washed with deionized water. 3 t of cheese whey (pH: 6.2) that had been heat-sterilized at 121° C. for 30 seconds was passed through the column at a flow rate of 10 l/min. The column was then sufficiently washed with deionized water, and the basic protein fraction adsorbed on the resin was eluted with a 0.1M citrate buffer solution (pH: 5.7) containing 0.9M sodium chloride. The eluate was desalted and concentrated by electrodialysis (ED), and freeze-dried to obtain 183 g of powdery basic protein fraction. The basic protein fraction may be directly used as the fracture repair promoter of the invention.

### Example 3

A column (diameter: 100 cm, height: 20 cm) filled with 50 kg of acidic polysaccharide gel (carrageenan) that had been processed into beads (see JP-A-61-246198) was sufficiently washed with deionized water. 3000 liters of skimmed milk (pH: 6.7) was passed through the column at a flow rate of 25 ml/min. The column was then sufficiently washed with deionized water, and the basic protein fraction adsorbed on the resin was eluted with a 0.02M carbonate buffer solution (pH: 7.0) containing 1.5M sodium chloride. The eluate was desalted and concentrated using a reverse osmosis (RO) membrane, and freeze-dried to obtain 136 g of powdery basic protein fraction. The basic protein fraction may be directly used as the fracture repair promoter of the invention.

### Example 4

50 g of the basic protein fraction obtained in Example 1 was dissolved in 10 liters of distilled water. After pepsin (manufactured by Kanto Kagaku Co., Ltd.) was added thereto so as to have a concentration of 2%, the basic protein fraction was hydrolyzed at 37° C. for 1 hour with stirring. After adjusting the pH of the mixture to 6.8 using a sodium hydroxide solution, 1% pancreatin (manufactured by Sigma) was added to the mixture. The mixture was then reacted at 37° C. for 2 hours. After the reaction, the protease was inactivated by heating the mixture at 80° C. for 10 minutes, and 48.3 g of basic peptide fraction was obtained. The basic peptide fraction may be directly used as the fracture repair promoter of the invention.

### Example 5

40 g of the basic protein fraction obtained in Example 2 was dissolved in 8 liters of distilled water. After trypsin (manufactured by Kanto Kagaku Co., Ltd.) was added thereto so as to have a concentration of 2%, the basic protein fraction was hydrolyzed at 37° C. for 1 hour with stirring. After adjusting the pH of the mixture to 6.6 using a sodium hydroxide solution, 1% pancreatin (manufactured by Sigma) was added to the mixture. The mixture was then reacted at 37° C. for 2 hours. After the reaction, the protease was inactivated by heating the mixture at 80° C. for 10 minutes, and 38.6 g of basic peptide fraction was obtained. The basic peptide fraction may be directly used as the fracture repair promoter of the invention.

### Test Example 5

### Animal Experiments

Animal experiments were performed using the basic protein fraction obtained in Example 1.

6-week-old male mice (C3H/HeJ) were used for the experiments. Each mouse was anesthetized by inhalation of diethyl ether, and pentobarbital was intraperitoneally administered to the mouse under anesthesia. The front portion on the left tibia of the mouse was shaved, disinfected, dissected to a length of 15 mm, and bluntly peeled to expose the tibia. The tibia was then cut off in the direction perpendicularly intersecting the longitudinal direction using a diamond disk at a position 5 mm under the patellar ligament to produce a bone fracture. After reposition, a 25 G needle was inserted into the intraspinal space, and fixed. The muscle and the skin were sutured using a 4-0 silk thread. A needle was inserted into the intraspinal space of the right tibia without causing a fracture, (this group was named “pseudo-operation group”). After confirming awakening, the basic protein fraction was started to administer to the mouse. The animal experiments were conducted on the non-administration group (CTRL), the 0.165% administration group, and the 1% administration group. The basic protein fraction was dissolved in drinking water, and orally administered. The basic protein fraction was replaced every two days in order to prevent putrefaction.

The fracture repair state was evaluated as follows: after 4 weeks from the operation, the mouse was subjected to perfusion fixation under deep anesthesia, and a soft X-ray image (Softex, Tokyo) and a μCT image were photographed.

As biomechanics analysis, the mechanical strength of the tibia after 4 weeks from the operation was determined using a precision universal testing machine with electronic measurement and control system (autograph). The total energy (toughness: resistance until fracture occurs, i.e. total area of stress-strain curve) was evaluated.

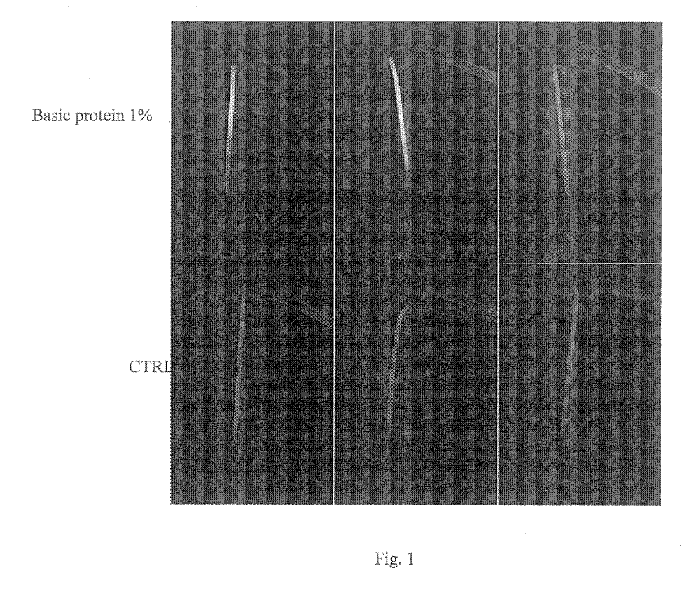

The results are shown in FIGS. 1 to 3.

FIG. 1 shows the soft X-ray images of the mouse fracture models in the basic protein fraction non-administration group (CTRL) and the 1% administration group (after 4 weeks from the operation).

As shown in FIG. 1, it was clearly found that the basic protein fraction administration group had a bone density higher than that of the control group around the fracture line.

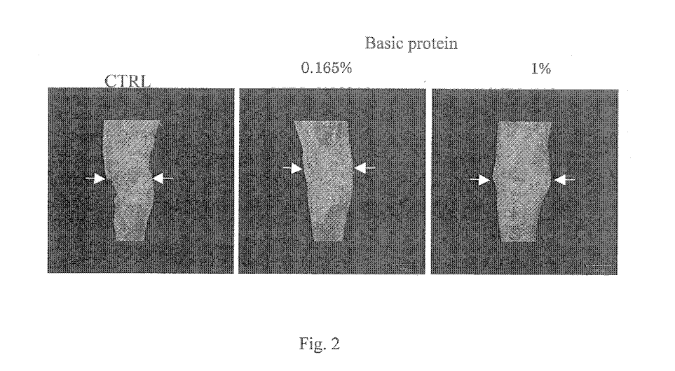

FIG. 2 shows the μCT images of the mouse fracture models.

An area having a high bone mineral density (BMD) is indicated by a warm color, and an area having a low BMD is indicated by a cold color. The fracture site is indicated by white arrows. In the basic protein fraction administration group, an area around the fracture site had a high BMD. This clearly indicates that repair of the fracture tended to be promoted.

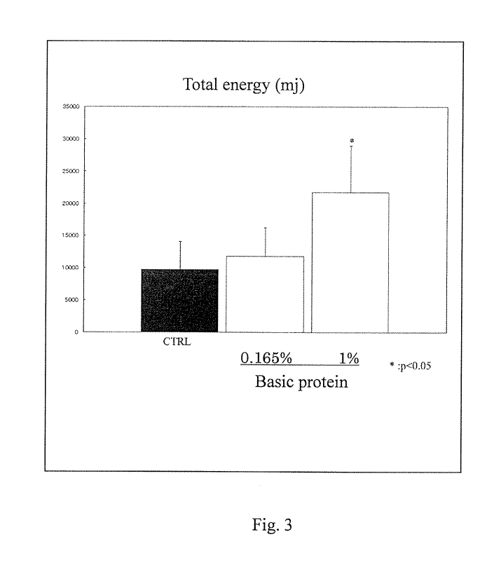

FIG. 3 shows the total energy that indicates the toughness of the bone.

It was confirmed that the total energy of the basic protein fraction administration group increased as compared with that of the basic protein fraction non-administration group (CTRL). Further, the 1% basic protein fraction administration group showed a significantly high total energy value. It was thus confirmed that repair of the fracture was promoted in the basic protein fraction administration group as compared with the non-administration group.

Note that similar effects were observed when using the basic peptide fraction obtained in Examples 4 and 5 (but, experimental results are not shown here).

### Example 6

The components shown in Table 4 were mixed, and formed under pressure to produce a tablet containing the milk-derived basic protein fraction obtained in Example 1 and having a fracture repair-promoting effect.

TABLE 4

| Hydrous crystalline glucose | 59.4 (wt %) |
|---|---|
| Basic protein fraction (Example 1) | 16.0 |
| Corn starch | 12.0 |
| Cellulose | 4.0 |
| Corn oil | 4.0 |
| Vitamin mixture (including choline) | 1.0 |
| Mineral mixture | 3.6 |

### Example 7

The components shown in Table 5 were mixed, put in a container, and heat-sterilized to produce a drink containing the milk-derived basic protein fraction obtained in Example 2 and having a fracture repair-promoting effect.

TABLE 5

| Mixed isomerized sugar | 15.0 (wt %) |
|---|---|
| Fruit juice | 10.0 |
| Citric acid | 0.5 |
| Basic protein fraction (example 2) | 0.5 |
| Essence | 0.1 |
| Calcium | 0.1 |
| Water | 73.8 |

### Example 8

The components shown in Table 6 were mixed, put in a container, and heat-sterilized to produce a jelly containing the milk-derived basic protein fraction obtained in Example 1 and having a fracture repair-promoting effect.

TABLE 6

| Fructose | 20.0 (wt %) |
|---|---|
| Granulated sugar | 15.0 |
| Glutinous starch syrup | 5.0 |
| Agar | 1.0 |
| Basic protein fraction (Example 1) | 0.5 |
| Essence | 0.1 |
| Calcium | 0.1 |
| Water | 58.3 |

### Example 9

The components shown in Table 7 were mixed, and emulsified at 85° C. to produce a processed cheese containing the milk-derived basic protein fraction obtained in Example 1 and having a fracture repair-promoting effect.

TABLE 7

| Gouda cheese | 43.0 (wt %) |
|---|---|
| Cheddar cheese | 43.0 |
| Sodium citrate | 2.0 |
| Basic protein fraction (Example 1) | 0.5 |
| Milk-derived calcium | 1.0 |
| Water | 10.5 |

### Example 10

The components shown in Table 8 were mixed to produce dough. The dough was baked to produce a cookie containing the milk-derived basic protein fraction obtained in Example 2 and having a fracture repair-promoting effect.

TABLE 8

| Flour | 50.0 (wt %) |
|---|---|
| Sugar | 20.0 |
| Salt | 0.5 |
| Margarine | 12.5 |
| Egg | 12.1 |
| Water | 2.9 |
| Sodium hydrogen carbonate | 0.1 |
| Ammonium bicarbonate | 0.2 |
| Calcium carbonate | 0.5 |
| Basic protein fraction (Example 2) | 1.2 |

### Example 11

The components shown in Table 9 were mixed to produce a dog food containing the milk-derived basic protein fraction obtained in Example 1 and having a fracture repair-promoting effect.

TABLE 9

| Soybean meal | 12.0 (wt %) |
|---|---|
| Skimmed milk powder | 14.0 |
| Soybean oil | 4.0 |
| Corn oil | 2.0 |
| Palm oil | 28.0 |
| Corn starch | 15.0 |
| Flour | 8.0 |
| Wheat bran | 2.0 |
| Vitamin mixture | 9.0 |
| Mineral mixture | 2.0 |
| Cellulose | 3.0 |
| Basic protein fraction (Example 1) | 1.0 |

1. A fracture repair promoter comprising a milk-derived basic protein fraction as an active ingredient.

2. The fracture repair promoter according to claim 1, wherein the milk-derived basic protein fraction comprises basic amino acids in an amount of 15 wt % or more to the total amino acids.

3. A fracture repair promoter comprising a basic peptide fraction obtained by hydrolyzing the milk-derived basic protein fraction according to claim 1 using a protease as an active ingredient.

4. The fracture repair promoter according to claim 3, wherein the protease is at least one protease selected from the group consisting of pepsin, trypsin and chymotrypsin.

5. The fracture repair promoter according to claim 3, wherein the protease is at least one protease selected from the group consisting of pepsin, trypsin and chymotrypsin, and pancreatin.

6. A food or drink comprising the milk-derived basic protein fraction or the basic peptide fraction according to claim 1.

7. A feed comprising the milk-derived basic protein fraction or the basic peptide fraction according to claim 1.

8. A method of producing a fracture repair promoter comprising bringing milk or a milk-derived raw material into contact with a cation-exchange resin to adsorb basic proteins on the cation-exchange resin, eluting the fraction adsorbed on the cation-exchange resin using an eluant having a salt concentration of 0.1 M to 1.0M, and using the eluted fraction as an active ingredient.

9. A method of producing a fracture repair promoter comprising bringing milk or a milk-derived raw material into contact with a cation-exchange resin to adsorb basic proteins on the cation-exchange resin, eluting the fraction adsorbed on the cation-exchange resin using an eluant having a salt concentration of 0.1 M to 1.0M, hydrolyzing the eluted fraction using a protease, and using the fraction obtained through hydrolyzing step as an active ingredient.

10. The method according to claim 9, wherein the protease is at least one protease selected from the group consisting of pepsin, trypsin and chymotrypsin.

11. The method according to claim 9, wherein the protease is at least one protease selected from the group consisting of pepsin, trypsin and chymotrypsin, and pancreatin.
